(19) United States
(12) Patent Application Publication
(10) Pub. No.: US 2020/0323516 A1
(43) Pub. Date: Oct. 15, 2020

(54) Three-Dimensional Dynamic Contrast Enhanced Ultrasound and Real-Time Intensity Curve Steady-State Verification during Ultrasound-Contrast Infusion

(71) Applicant: The Board of Trustees of the Leland Stanford Junior University, Stanford, CA (US)

(72) Inventors: Ahmed El Kaffas, Palo Alto, CA (US); Dimitre H. Hristov, Stanford, CA (US); Juergen K. Willmann, Stanford, CA (US)

(21) Appl. No.: 16/844,701

(22) Filed: Apr. 9, 2020

Related U.S. Application Data
(60) Provisional application No. 62/831,505, filed on Apr. 9, 2019.

(51) Int. Cl. A61B 8/08; A61B 8/00; A61M 5/00; A61M 5/172

(52) U.S. Cl. CPC A61B 8/481; A61B 8/5246; A61B 8/4245; A61B 2090/365; A61B 8/46; A61M 5/007; A61M 5/172; A61B 8/463

(57) ABSTRACT

Guidance and visualization for three-dimensional dynamic contrast-enhanced ultrasound imaging is provided. Anatomical B-mode images and live three-dimensional dynamic contrast-enhanced ultrasound images (3D-DCE-US) of an anatomical region of interest are acquired. An ultrasound imaging probe tracker tracks six degrees of freedom position and orientation data of the ultrasound imaging probe. With reference to the common three-dimensional coordinate frame, and for each of the acquired images, the anatomical B-mode images, the live 3D-DCE-US images, and a three-dimensional computer-generated model are visualized and overlaid with each other. The visualization provides guidance and feedback to a user of the ultrasound imaging probe during three-dimensional dynamic contrast-enhanced ultrasound imaging.

### CROSS-REFERENCE TO RELATED APPLICATIONS

This application claims priority from U.S. Provisional Patent Application 62/831505 filed Apr. 9, 2019, which is incorporated herein by reference.

### FIELD OF THE INVENTION

This invention relates to methods for three-dimensional dynamic contrast-enhanced ultrasound imaging and interventions.

### BACKGROUND OF THE INVENTION

Ultrasound (US) is amongst the most widely accessible medical imaging modalities. In comparison to MRI or CT, it offers inexpensive bedside diagnostics without radiation and/or contrast restrictions related to kidney failure. As a result, functional ultrasound using 1D array transducers has been proposed as an ideal tool for longitudinal imaging applications such as cancer treatment monitoring to assess early tumor perfusion changes as surrogate for response.

An important limitation in longitudinal applications of ultrasound is positioning the transducer to obtain the same imaging plane for day-to-day comparison. In dynamic contrast-imaging protocols, the transducer must remain still in the imaging plane of choice. This is especially problematic in cancer imaging, where tumors tend to be highly heterogeneous, on a plane-to-plane basis. As a result, the operator-dependent nature of ultrasound can result in erroneous quantitative measurements and difficulties in navigating a transducer to different anatomical sites during time-sensitive scan sessions to capture different 2D planes.

The use of Three-Dimensional Dynamic Contrast-Enhanced Ultrasound (3D DCE-US) using matrix transducers for longitudinal imaging applications has been suggested and recently demonstrated as means to overcome the above-mentioned challenges by capturing whole-tumor perfusion simultaneously. However, several major challenges remain in the use of 3D DCE-US. One specifically relates to probe motion during acquisition, which can be imposed by either the operator or the patient. This can affect quantification and requires motion correction. However, unlike conventional 2D contrast modes in clinical systems, current commercially available 3D DCE-US modes do not display a low mechanical index B-mode and contrast-mode image side-by-side, thus leaving the operator with no positioning feedback during lengthy acquisitions to capture the dynamic contrast signal.

In addition, without stable anatomical features from B-mode images, post-acquisition motion correction can be a significant challenge. As a result, the quality of lengthy acquisitions (>30 seconds) to capture the temporal contrast dynamics during a bolus (2-4 min from wash-in to wash-out) or disruption-replenishment (>4 min) acquisition can be compromised.

The present invention addresses these problems of position feedback and motion correction in 3D DCE-US.

### SUMMARY OF THE INVENTION

A method and system for providing guidance and visualization for three-dimensional dynamic contrast-enhanced ultrasound imaging is disclosed. Anatomical B-mode images of an anatomical region of interest are acquired by an ultrasound imaging probe. Live three-dimensional dynamic contrast-enhanced ultrasound images (3D-DCE-US) of the anatomical region of interest are also acquired by the ultrasound imaging probe. An ultrasound imaging probe tracker is used for tracking six degrees of freedom position and orientation data of the ultrasound imaging probe during and for each of the respective acquired images. A computer implemented code operable on a computer system (i) determines a common three-dimensional coordinate frame obtained from the six degrees of freedom position and orientation data of the ultrasound imaging probe, and generates a three-dimensional computer-generated model of the ultrasound imaging probe. The computer implemented code further (i) visualizes and overlayes with each other with reference to the common three-dimensional coordinate frame, and for each of the acquired images, the (j) the anatomical B-mode images, (jj) live 3D-DCE-US images, and (jjj) the three-dimensional computer-generated model. The visualization then provides guidance and feedback to a user of the ultrasound imaging probe during three-dimensional dynamic contrast-enhanced ultrasound imaging.

In one example, the visualization could be performed by an augmented reality system.

In one example, the computer implemented code generates and feeds back to the user positioning differences or errors of the actual position of the ultrasound imaging probe relative to the target position of the ultrasound imaging probe. In another example, the computer implemented code generates corrective actions regarding the position of the ultrasound imaging probe. Feedback could be visual feedback, audible feedback or haptic feedback.

In still another example, the computer implemented code quantifies and visualizes a signal intensity within the anatomical region of interest. The signal intensity allows the user to adjust an infusion rate. The signal intensity could also allow the user to adjust an infusion rate in an infusion during the three-dimensional dynamic contrast-enhanced ultrasound imaging. Further, the signal intensity could allow the user to determine a steady-state in an infusion during the three-dimensional dynamic contrast-enhanced ultrasound imaging and triggering microbubble destruction pulses.

### BRIEF DESCRIPTION OF THE DRAWINGS

FIGS. 1-2 show according to exemplary embodiments of the invention acquisition methods and systems for three-dimensional dynamic contrast enhanced ultrasound and real-time intensity curve steady-state verification during ultrasound-contrast infusion.

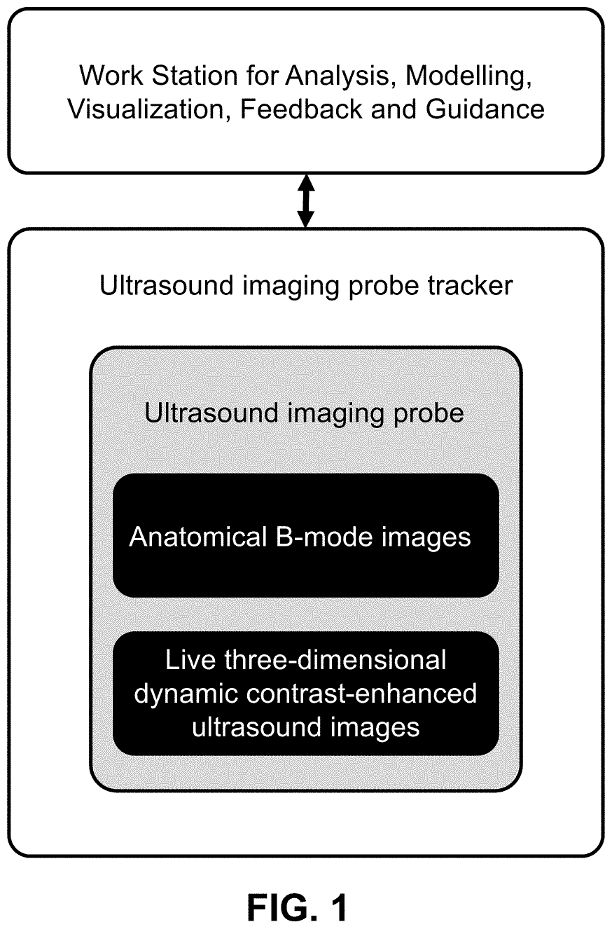

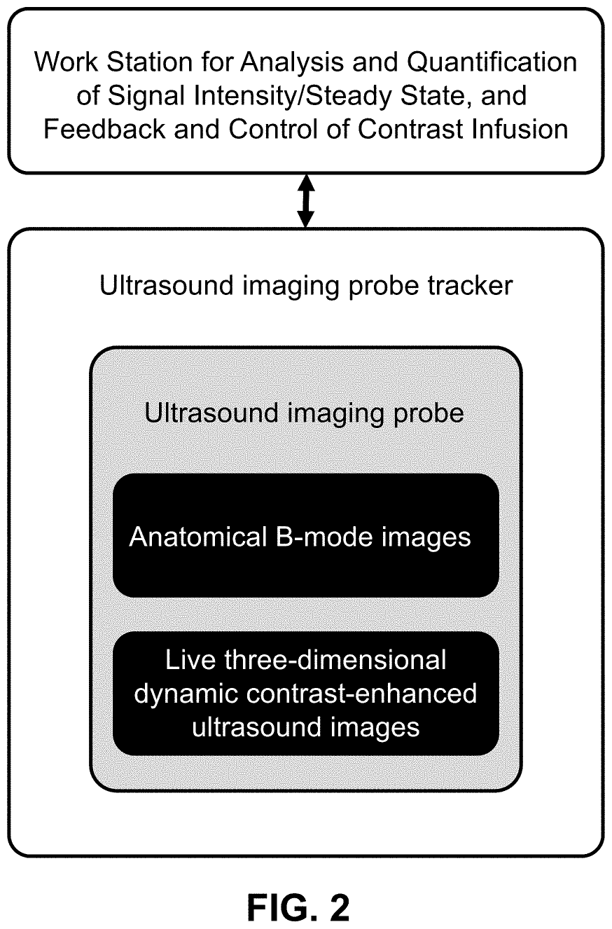

### DETAILED DESCRIPTION

Continuous positioning feedback such as available in 2D, would potentially improve the quality of the data for post-processing and quantification, by ensuring that the operator maintains a steady position throughout a scan. The inventors developed an interventional acquisition system for 3D DCE-US imaging that aims to provide users with navigation feedback and temporal transducer coordinates to re-align the imaging planes in 4D for post-processing and quantification. The interventional system was also designed with disruption-replenishment (DR) imaging in mind, which has been shown to be more repeatable and quantitative over conventional bolus-based acquisitions.

In brief, DR uses short high-power ultrasound pulses (disruptions within diagnostic range) to momentarily burst microbubbles flowing at steady state. The rate of replenishment of microbubbles is then modeled to extract quantitative parameters. The advantages of DR obviate the need to estimate the indicator input function. A key requirement for contrast used is further decreasing potential risks linked to the procedure as well length of the procedure.

For the purposes of this invention the feasibility was determined of using optical tracking-based navigation assistance to provide positioning feedback in scenarios where no positioning feedback is provided. More specifically, transducer displacement during a lengthy acquisition was assessed under blind and B mode-guided scenarios and compared to navigation assisted positioning. For a subset of patients, it was also evaluated if 4D image re-alignment improves quantification repeatability.

Navigation System

The optical tracking system was designed and developed in house to facilitate transducer navigation, and to help maintain a specific imaging location over an anatomical site to overcome the lack of side-by-side contrast and B-mode image display in current commercial implementations of 3D DCE-US. The system displays on an LCD screen a live virtual probe that moves (translation and rotation) in sync with a real world (real time) optically tracked transducer. The system also captures reference coordinates and displays a second virtual probe in red at these coordinates to help operators maintain the same position and orientation of the transducer during lengthy scan sessions that can last up to 10 minutes.

In an exemplary embodiment, at the core of the platform is a modular research interventional workstation, which supports a Digital Navigation Link (DNL) for data transfer from a Philips EPQ7 (Philips, Bothell, Wash.) ultrasound system. Over a 1 Gb/s Ethernet connection, the DNL allows real-time transfer of 2D and 3D images from the scanners to the interventional workstation, which immediately displays them in real-time with user selectable layouts. Within the interventional workstation, interfaces and modules have been implemented and validated to link ultrasound images to an external (world) coordinate system through optical cameras, which have a stereo pair of infrared (IR) cameras (Polaris, NDI, Canada) set-up over the patient bed. Before each abdominal scan session, the cameras are oriented to face the lower-mid section of the abdomen using a laser pointer mounted at a mid-point between both cameras. The transducer was equipped with an in-house 3D-printed contraption for holding four reflective spheres that are uniquely seen and tracked by the dual IR cameras (Polaris, NDI, Canada), and uniquely visible to the infrared cameras. The tool is set-up in such a way as to allow tracking translational and rotational movements of the transducer. For the work presented here, the tool was attached to an X6-1 (Philips, Bothell, Wash.) matrix transducer. The infrared cameras were connected to a PC running windows 7 and the MevisLab (Mevis Medical Solutions AG, Bremen, Germany) software package. An example is shown in the figure on page 2 of Appendix B in U.S. Provisional Patent Application 62/831505 filed Apr. 9, 2019, which is incorporated herein by reference.

The MevisLab platform was used for real-time processing of incoming optical tracking data, acquisition of volumetric ultrasound data, and visualization of ultrasound data and virtual probes. A custom software was developed to handle these tasks. The software was developed using customized MevisLab modules written in C++ and Python. The user interface was implemented using MevisLab's custom MDL language with additional Python scripting. The software runs on the interventional computer workstation and communicates with the EPIQ7 ultrasound device and the Polaris optical tracking system. A 3D virtual model of the transducer with tracking tool was created in MevisLab with a specialized graphics unit, and linked to the streaming tracking coordinates through transformation matrices, which uniquely leverages the volumetric nature of 3D US (i.e. X6-1). Calibration of the method was based on the “hand-eye” technique. The method is fully automated with the use of data rejection based on sensor displacements, automatic registration of overlapping image regions, and a self-consistency error metric evaluated continuously during the calibration process. As a result, the uncertainty in spatial localization of US image features is smaller than 1.5 mm.

All imaging evaluations were carried out using a clinical EPIQ7 ultrasound system coupled to a clinical X6-1 transducer (Philips Healthcare, Andover, Mass.). The X6-1 2D matrix transducer has 9212 elements that steer the US beam in real-time using a micro-beam former located in the transducer head, and enables up to 90 degrees by 90 degrees wide volumes. It was designed for abdominal applications, with a frequency range of 1-6 MHz (center frequency, 3.2 MHz) (32). All contrast data was acquired in volumetric (3D) contrast-specific imaging (contrast-mode) with a low mechanical index (MI=0.09) to allow non-destructive visualization of microbubbles. Disruption-replenishment used 3 flash frames with an MI of 0.77.

Evaluation of Operator Performance with Navigation

Five experienced ultrasound operators were recruited to test the system in a clinical scenario reminiscent of a 3D DCE-US imaging session where long imaging sequences need to be acquired. To do so, sonographers' ability was tested to return to a reference position, or maintain the transducer in the same location with access to different sources of feedback. Each operator had at least 2 years of ultrasound imaging experience. Sonographers were first given up to 10 minutes to familiarize themselves with the tracking system and virtual probe display. Sonographers were then asked to locate an abdominal landmark using conventional B-mode images within a healthy volunteer.

Once landmarks were located, operators were first asked to remove the transducer and return the exact same anatomical location on the abdomen. The repositioning error and time to return to the reference position were recorded as metrics. This was repeated for each operator twice. Operators were then asked to maintain the transducer position for 4 min under three feedback methods: i) conventional B-mode, ii) display of a real-time virtual probe and a reference virtual probe placed at the original location used to identify the abdominal landmark, iii) blind (no B-mode or virtual probe display). For the testing, sonographers used an X6-1 transducer with tracking attachment, connected to an EPIQ7 system (Philips, Bothell, Wash.).

To quantify results, the magnitude of displacement of a center voxel in the image over the cine (acquisition sequence) was computed relative to the reference position as an estimate of the imaging position error. Histograms of displacements throughout the whole cine were also generated in order to examine the extent and directionality of displacement using histogram features such as the mean, median, standard deviation (S.D.) and skewness of the bins.

Patient Inclusion for Evaluation in Clinical 3D DCE-US

Clinical 3D DCE-US patient data was obtained from an ongoing HIPPA compliant longitudinal prospective study approved by the Institutional Review Board of our institution and written consent was obtained from all participating patients. All patients were imaged with disruption-replenishment imaging with repeated pairs of disruption ‘flash’ events over an 8-minute infusion. The data was used to assess repeatability of quantitative 3D DCE-US parameters with and without tracking, and to evaluate the need for a live TIC tool. For this purpose, 8 adult (>18 years old) patients with a total of 14 3D DCE-US scans were prospectively included. All patients with liver metastases ≥1 cm in diameter based on CT or MR imaging were considered eligible for our study and a clinical oncologist referred them to our study after introducing the study to the patients. Patients with documented anaphylactic or other severe reaction to any contrast media; pregnant or lactating patients; and patients with cardiac shunts or presence of severe pulmonary hypertension (all are contraindication for ultrasound contrast agent) were excluded. No patient was excluded due to exclusion criteria. Five patients were women (mean age, 54.5 years; range, 48-60 years) and 6 patients were men (mean age, 57.6 years; range, 47-68 years). Included patients had liver metastases originating from the following primary tumors: rectal adenocarcinoma (n=2); pancreatic adenocarcinoma (n=1); pancreatic neuroendocrine tumor (n=4); and colonic adenocarcinoma (n=4).

Dynamic Contrast-Enhanced Ultrasound Imaging: Contrast Agent Administration, Acquisition and Quantification

Clinical contrast microbubbles (Definity; Latheus Medical Imaging, North Billerica, Mass.; FDA-approved for echocardiography and administered off-label for liver imaging) were used. These microbubbles are perfluorobutane lipid microspheres with a mean diameter of 1.8 μm (range 1-10 μm). The DR DCE-US acquisition methods was used. Patients were infused for up to 3 min with a solution of 0.9 ml of the contrast agent mixed in 35.1 ml of saline at a constant rate of 0.08 ml/s (15) using a syringe pump (Medfusion 3500; Smiths Medical, Dublin Ohio) to reach steady state which was confirmed using an in-house developed time intensity curve (TIC) tool displaying TIC in real time on a separate monitor. Two disruption-replenishments sequences (R1 and R2) were applied (2.5 minutes apart) with the contrast agent continuously infused to assess repeatability. During the replenishment time (at destruction-replenishment DCE-US), all patients were asked to either hold their breath (for up to 30 seconds) or to breathe shallow (in patients unable to hold their breaths) to minimize motion artifacts. Examples are shown in the figure on page 4 of Appendix B in U.S. Provisional Patent Application 62/831505 filed Apr. 9, 2019, which is incorporated herein by reference.

Image analysis of 3D DCE-US was carried-out by one reader in random order using software developed in-house in MevisLab (MevisLab, Germany) and MATLAB (Mathworks, Mass., USA). A volume-of-interest (VOI) was delineated by covering the entire liver lesion volume viewed on axial, sagittal, and coronal imaging planes. This VOI was subsequently used to generate TICs for the quantification of perfusion as described below. Post-processing and analysis steps for DCE-US following VOI selection consisted of the following steps: (1) linearization of the US image voxel values in contrast-mode images using a transformation function and a compression parameter provided by the equipment manufacturer; (2) extraction of TIC proportional to contrast concentration from the average signal intensity in the VOI; (3) standardized monoexponential fitting to VOI average intensity (TIC) using standard quantitative models in Matlab. The custom analysis software permitted the selection of VOIs around the lesion, and the application of dynamic enhancement models that could not otherwise be applied in 3D by using commercial software. Monoexponential curve fitting was performed from the first frame after the disruption event for a minimum of 1 min for DR DCE. The following parameters were extracted: relative blood volume (rBV), and relative blood flow (rBF). The rBV is proportional to blood volume; the rBF is proportional to flow/perfusion rate. Examples are shown in the figure on page 3 of Appendix B in U.S. Provisional Patent Application 62/831505 filed Apr. 9, 2019, which is incorporated herein by reference.

Statistical Analysis

To measure repeatability of DCE-US quantitative parameters from DR DCE-US data sets, pairs of log-transformed measurements were assessed by intraclass correlation coefficient (ICC) from a random-effects model, with random effects of scan session (SS) nested within patient. Log-transformation was applied to make the data normally distributed for standard statistical analysis. The 95% confidence intervals (CI) was also calculated for each ICC. ICC of 0-0.20 indicated no agreement; ICC of 0.21-0.40, poor agreement; ICC of 0.41-0.60, moderate agreement; ICC of 0.61-0.80, good agreement; and ICC greater than 0.80, excellent agreement. All statistical analyses were performed using Stata Release 14.1 (StataCorp LP, College Station, Tex.). Statistical significance was fulfilled at P<0.05.

Results: Tracking-Assisted Positioning Assessment

Data indicates that the tracking system of this invention assists operators to return to, or maintain a transducer position during a lengthy acquisition session, such as those commonly employed in contrast-enhanced ultrasound, by providing positioning feedback through the use of virtual transducer display. Overall, sonographers found the use of a virtual display of the probe for positioning feedback clinically feasible.

Repositioning error and time to reposition for each sonographer repeated trials, specified for each of the feedback methods. It was noted that while repositioning under Bmode feedback offered the most rapid and accurate results, tracking-assisted repositioning offered slightly worst results. No feedback resulted in large repositioning errors.

The average relative displacement throughout the acquisition sequence was used as a metric to assess the three different positioning feedback methods.

The figure as shown on page 3 in Appendix B in U.S. Provisional Patent Application 62/831505 filed Apr. 9, 2019, which is incorporated herein by reference, exhibits the magnitude of the displacement of a center voxel and associated histogram over the 4 min acquisition for each of the feedback conditions, for two sonographers. Histogram featured (mean, median, S.D. and skewness) for each of the sonographers showed that an average displacement of 3.75 mm with standard deviation (S.D.) of 3.31 mm and displacement histogram skewness of −0.18 was noted when using B-mode as feedback. When blinded (no B-mode or virtual probe display), an average displacement of 4.58 mm (S.D. 2.65 mm; skewness 6.19) was noted. In contrast, the average displacement for tracking-feedback using a virtual probe was comparable to that from B-mode at 3.48 mm, with a smaller standard deviation and less skewness of the displacement histogram (S.D. 0.8 mm; skewness 0.09). One operator performed better with tracking than B-mode; one operator performed better blinded than with tracking and B-Mode. The use of tracking for positioning feedback resulted in more consistent (smaller S.D.) results, and a mean displacement that was comparable to B-mode- based positioning feedback.

Clinical Use of Interventional System

Overall, the interventional system is advantageous and useful for sonographers as it assists positioning and maintaining a position during blind scans, especially during contrast sequences after contrast clears through disruption.

To confirm the need for a live TIC tool, using the complete infusion acquisition sequence, the time it takes for the contrast intensity to reach steady state in 3 different tissue was calculated: the liver lesion, the liver parenchyma, and the portal vein. The time to steady state was presented as a box and whisker plot for all patient scans (see figure as shown on page 4 in Appendix B in U.S. Provisional Patent Application 62/831505 filed Apr. 9, 2019, which is incorporated herein by reference). Note the large variability in the time-to-steady-state, specifically in the liver and lesion parenchyma, which could be affected by general patient physiology and especially by the histology of the primary tumor of the metastasis that would differ on a patient-by-patient basis. Overall, the live TIC tool was helpful in determining timing of disruption events.

Further examples and embodiments of the method and system of the invention are shown in Appendix A in U.S. Provisional Patent Application 62/831505 filed Apr. 9, 2019, which is incorporated herein by reference.

What is claimed is:

1. A method for providing guidance and visualization for three-dimensional dynamic contrast-enhanced ultrasound imaging, comprising:
(a) acquisition of anatomical B-mode images of an anatomical region of interest, wherein the anatomical B-mode images are obtained by an ultrasound imaging probe;
(b) acquisition of live three-dimensional dynamic contrast-enhanced ultrasound images (3D-DCE-US) of the anatomical region of interest, wherein the live 3D-DCE-US images are obtained by the ultrasound imaging probe;
(c) an ultrasound imaging probe tracker for tracking six degrees of freedom position and orientation data of the ultrasound imaging probe during and for each of the respective images acquired in (a) and (b);
(d) computer implemented code operable on a computer system for:
(i) determining a common three-dimensional coordinate frame obtained from the six degrees of freedom position and orientation data of the ultrasound imaging probe, and
(ii) generating a three-dimensional computer-generated model of the ultrasound imaging probe,
(e) the computer implemented code for:
visualizing and overlaying with each other with reference to the common three-dimensional coordinate frame, for each of the acquired images:
(j) the anatomical B-mode images,
(jj) live 3D-DCE-US images, and
(jjj) the three-dimensional computer-generated model,
wherein the visualizing provides guidance and feedback to a user of the ultrasound imaging probe during three-dimensional dynamic contrast-enhanced ultrasound imaging.

2. The method as set forth in claim 1, wherein the visualization is performed by an augmented reality system.

3. The method as set forth in claim 1, wherein the computer implemented code generating and feeding back to the user positioning differences or errors of the actual position of the ultrasound imaging probe relative to the target position of the ultrasound imaging probe.

4. The method as set forth in claim 1, wherein the computer implemented code generating corrective actions regarding the position of the ultrasound imaging probe.

5. The method as set forth in claim 1, wherein the feedback is visual feedback, audible feedback or haptic feedback.

6. The method as set forth in claim 1, further comprising the computer implemented code quantifying and visualizing a signal intensity within the anatomical region of interest.

7. The method as set forth in claim 6, wherein the signal intensity allowing the user to adjust an infusion rate.

8. The method as set forth in claim 6, wherein the signal intensity allowing the user to adjust an infusion rate in an infusion during the three-dimensional dynamic contrast-enhanced ultrasound imaging.

9. The method as set forth in claim 6, wherein the signal intensity allowing the user to determine a steady-state in an infusion during the three-dimensional dynamic contrast-enhanced ultrasound imaging and triggering microbubble destruction pulses.
